(19) United States
(12) Patent Application Publication
(10) Pub. No.: US 2017/0290697 A1
(43) Pub. Date: Oct. 12, 2017

(54) SPLINTS AND RELATED METHODS OF USE

(71) Applicant: WHITSTAR, LLC, Bountiful, UT (US)

(72) Inventor: Casey W. Boardman, Fayetteville, NC (US)

(21) Appl. No.: 15/485,018

(22) Filed: Apr. 11, 2017

Related U.S. Application Data
(60) Provisional application No. 62/321,551, filed on Apr. 12, 2016.

(51) Int. Cl. A61F 5/058

(52) U.S. Cl. CPC A61F 5/05841; A61F 5/0585; A61F 5/05858

(57) ABSTRACT

A splint may be used to support or immobilize a body portion of a patient. Some splints may include one or more features to facilitate transport, placement, and/or manipulation of a splint. For example, some splints may be relatively lightweight, rugged, and in a compact configuration prior to use. Some splints may use radiolucent materials to permit x-ray examination of the relevant appendage without removal of the splint. Some splints may be used with other accessories or devices to facilitate immobilization of a limb and/or transport of the injured individual.

### RELATED APPLICATIONS

This application claims priority to U.S. Provisional Patent Application No. 62/321,551, titled “SPLINTS AND RELATED METHODS OF USE,” filed Apr. 12, 2016, which is hereby incorporated herein by reference in its entirety.

### TECHNICAL FIELD

This application relates generally to splints or other devices for immobilizing and/or supporting a portion of a body of a patient.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1A is a view of a splint, according to one embodiment of the present disclosure, in an expanded configuration.

FIG. 1B is a view of the splint of FIG. 1A where the splint has been rotated 180 degrees about an axis of symmetry of the splint relative to the configuration depicted in FIG. 1A.

FIG. 1C is a cross sectional view of the splint of FIG. 1A.

FIG. 2A is a side view of a leg that is supported by the splint of FIG. 1A in an expanded configuration and with an ankle/elbow/knee device.

FIG. 2B is a side view of a leg that is supported by the splint of FIG. 1A in an expanded configuration and without the ankle/elbow/knee device.

FIG. 2C is a side view of a leg that is supported by the ankle/elbow/knee device of FIG. 2A.

FIG. 2D is a side view of an arm that is supported by the splint of FIG. 1A in a partially expanded configuration.

FIG. 2E is a side view of an arm that is supported by the splint of FIG. 1A in an unexpanded configuration.

FIG. 2F is a side view of an arm that is supported by the ankle/elbow/knee device of FIG. 2A.

FIG. 2G is a side view of a leg bent at the knee supported by the ankle/elbow/knee device of FIG. 2A.

### DETAILED DESCRIPTION

It will be readily understood that the components of the embodiments as generally described and illustrated in the figures herein could be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

The phrase “coupled to” is used in its ordinary sense, and is broad enough to refer to any suitable coupling or other form of interaction between two or more entities, including mechanical, fluid, and thermal interaction. Two components may be coupled to each other even though they are not in direct contact with each other. The phrases “attached to” or “attached directly to” refer to interaction between two or more entities which are in direct contact with each other and/or are separated from each other only by a fastener of any suitable variety (e.g., a hook-and-loop element).

The terms proximal and distal are generally used in their ordinary sense. For example, the term proximal, when used with reference to a splint or limb, may refer to a portion of the splint or limb that is near or nearer to the point of limb attachment. When used in connection with a splint or splint component, the term proximal may be used to refer to the end or region of the splint or component nearest or nearer to the torso of the patient when the splint is in normal use. The term “hook-and-loop” is broad enough to refer to arrangements comprising not only hooks and loops, but also fasteners that include hooks on both faces of material to be fastened together.

Splints or other related appliances may be used to immobilize or support a body portion. For example, splints or other immobilization devices may be used to immobilize or support an injured (e.g., broken) arm or leg. The need for splints may be particularly acute in remote locations (e.g., mountains, wilderness, ski slopes, military expeditions, etc.), where the injured individual must be transported to receive further medical assistance.

In some embodiments, a splint may include a first side to be oriented toward the injured limb when splinting a leg (e.g., a leg orientation) and a second side to be oriented toward the injured limb when splinting an arm (e.g., an arm orientation). In some embodiments, a splint may be configured for use with other accessories to immobilize a joint, such as a wrist or ankle.

In some embodiments, a splint or splint assembly may be configured for compact storage. For example, in some embodiments, the splint may be rolled, folded, or otherwise compacted to reduce the space occupied by the splint. This compact configuration may facilitate portability and storage of the splint, thereby enabling use of the splint in remote or otherwise difficult-to-access locations. In some embodiments, components of the splint may be used to support an arm in a sling configuration.

In some circumstances, it is desirable to immobilize the joints above and below the site of an injury to prevent further damage from rotation or movement about the joint. For example, for a fracture of a radius and/or ulna, it may be advantageous to immobilize the elbow and the wrist.

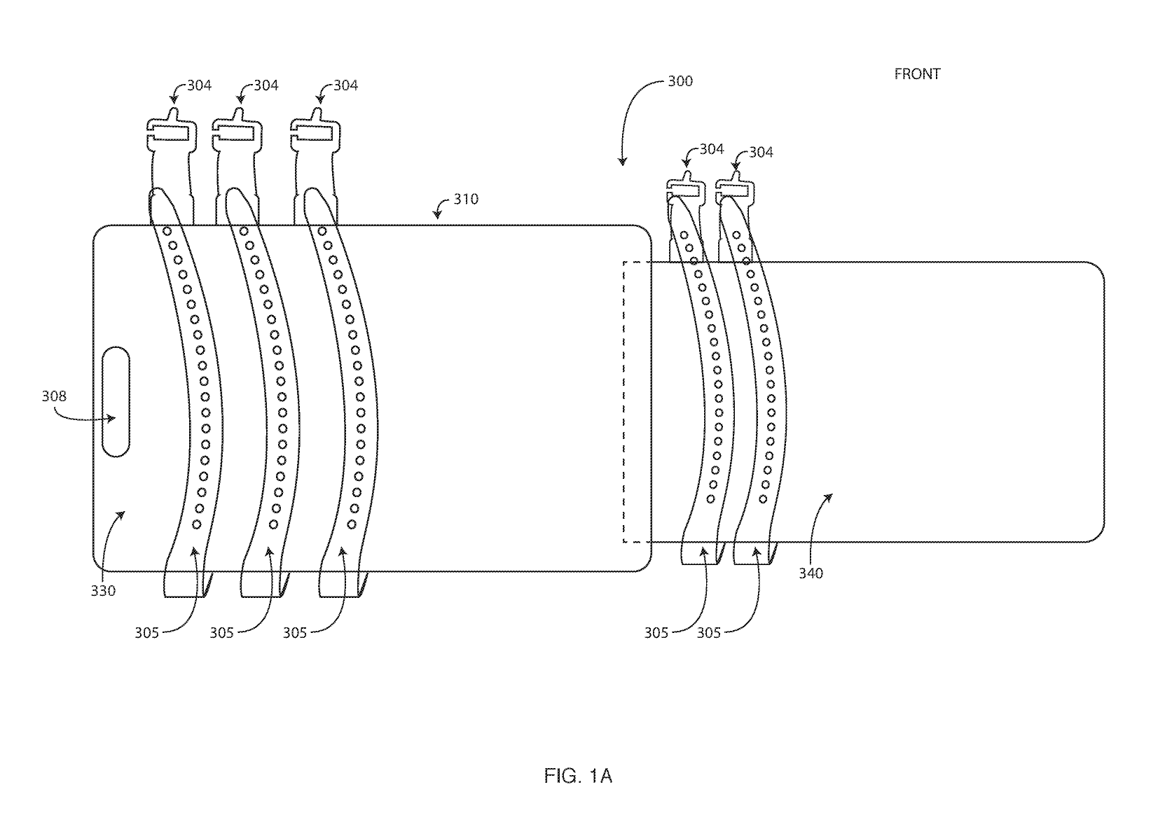

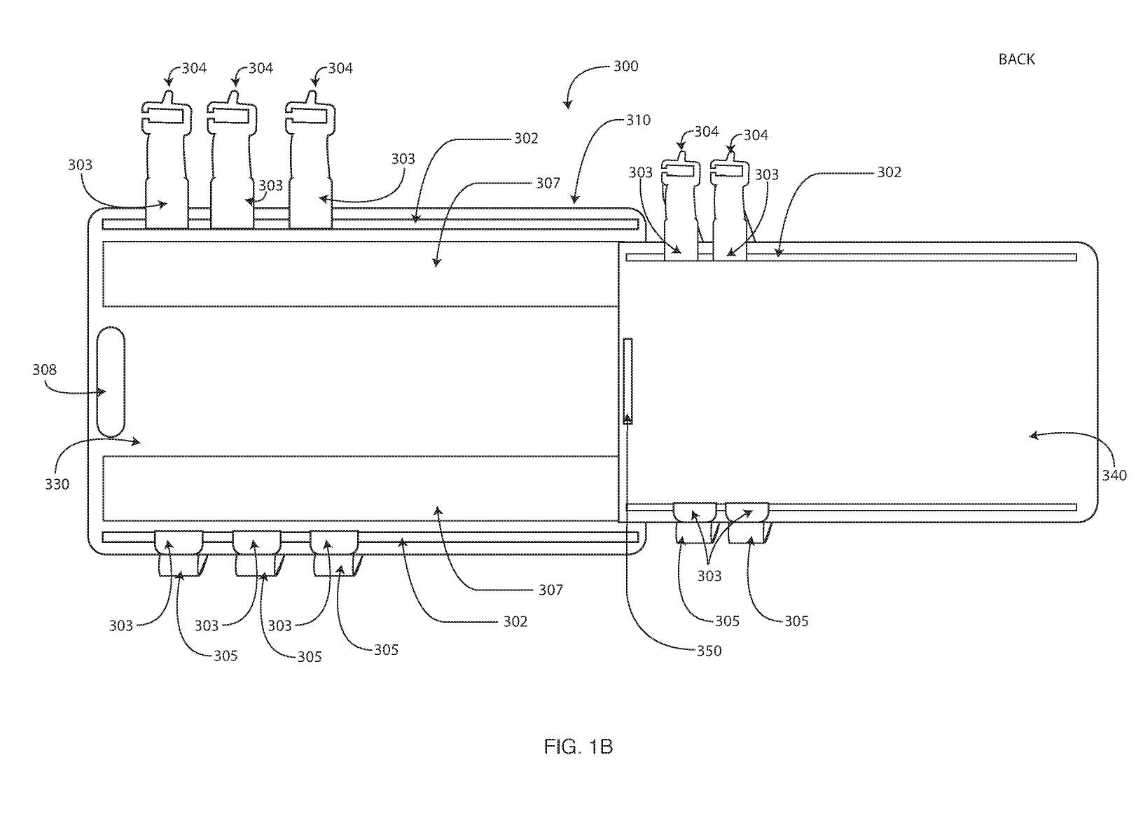

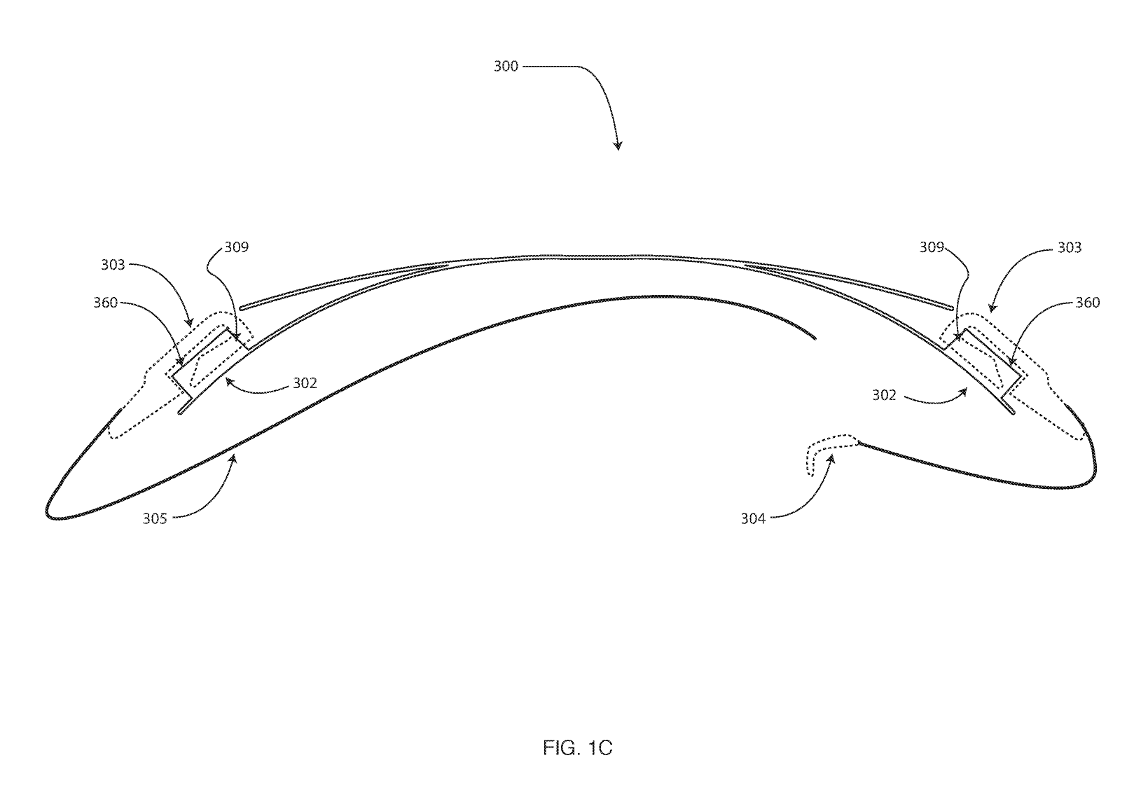

FIGS. 1A and 1B provide views of a splint 300, according to another embodiment of the present disclosure. FIG. 1A is a view of a splint 300 in an expanded configuration. FIG. 1B is a view the splint 300 where the splint 300 has been rotated 180 degrees about an axis of symmetry of the splint 300 relative to the configuration depicted in FIG. 1A, showing the rear or underside of the splint 300. FIG. 1C is cross sectional view of the splint 300 of FIG. 1A.

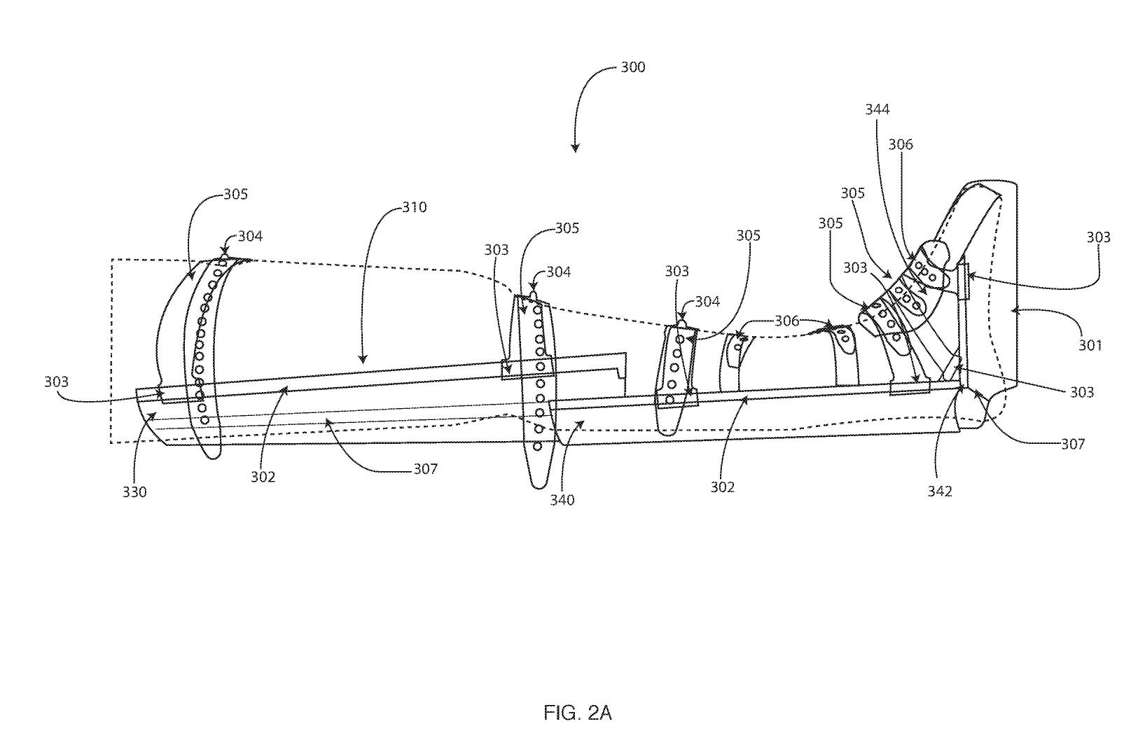

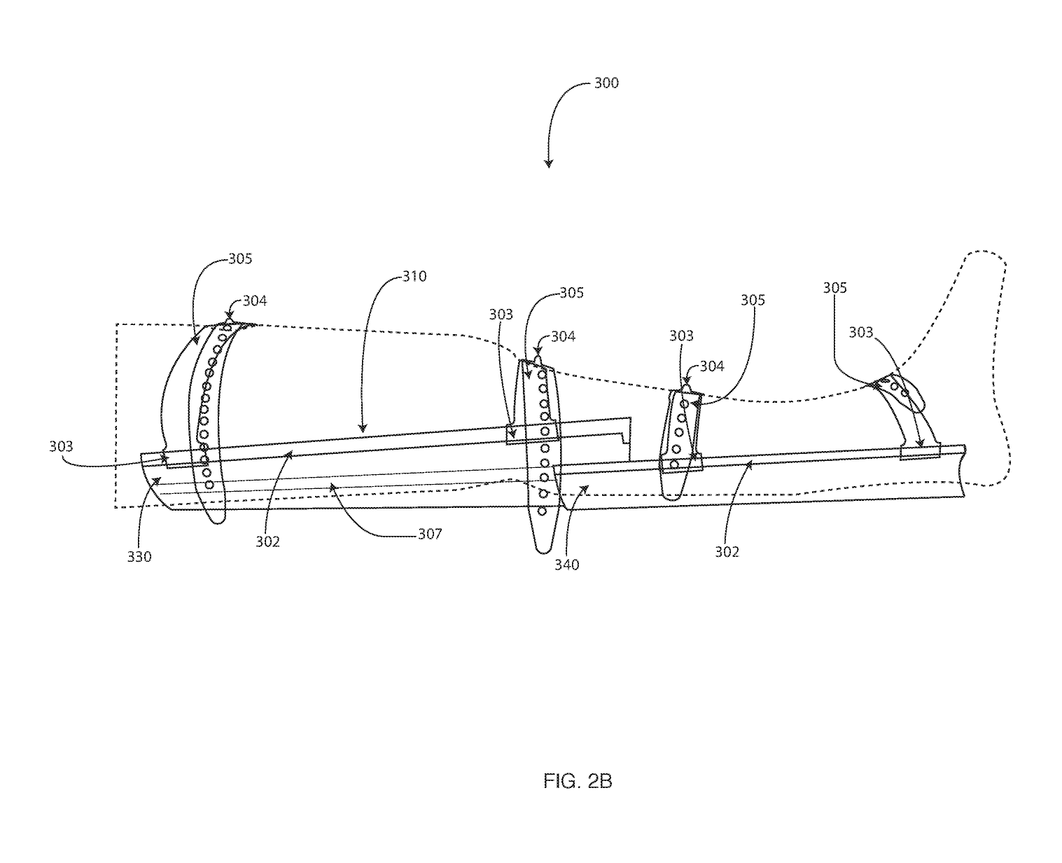

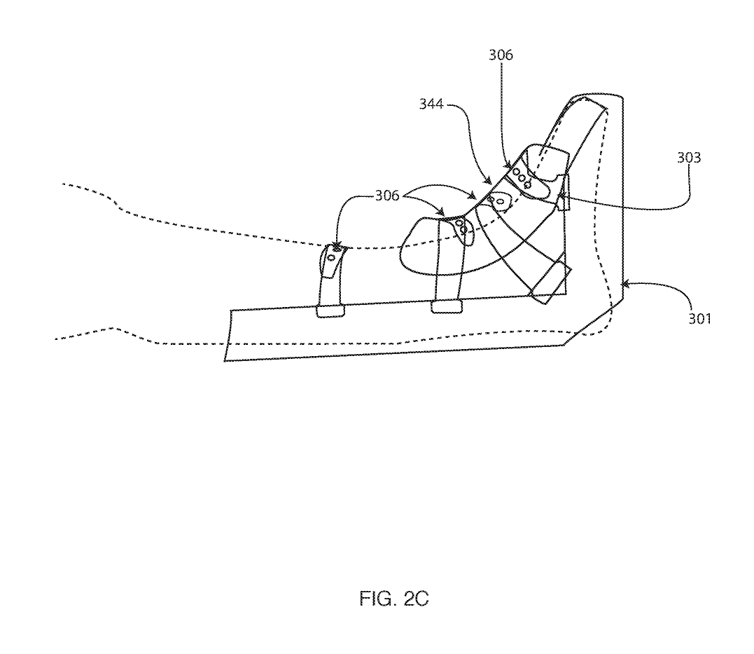

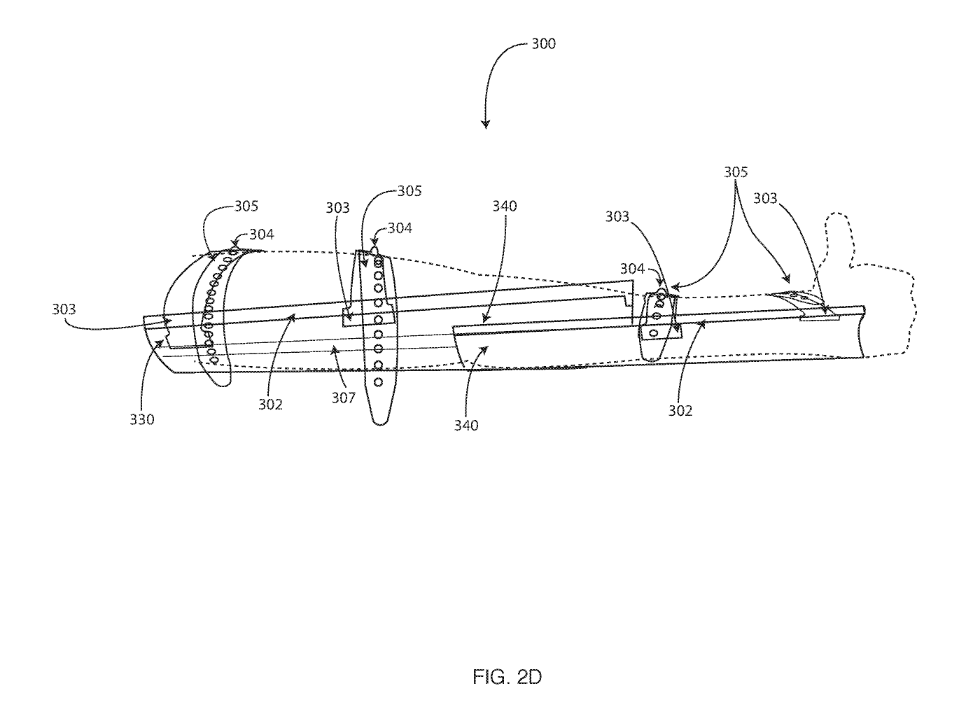

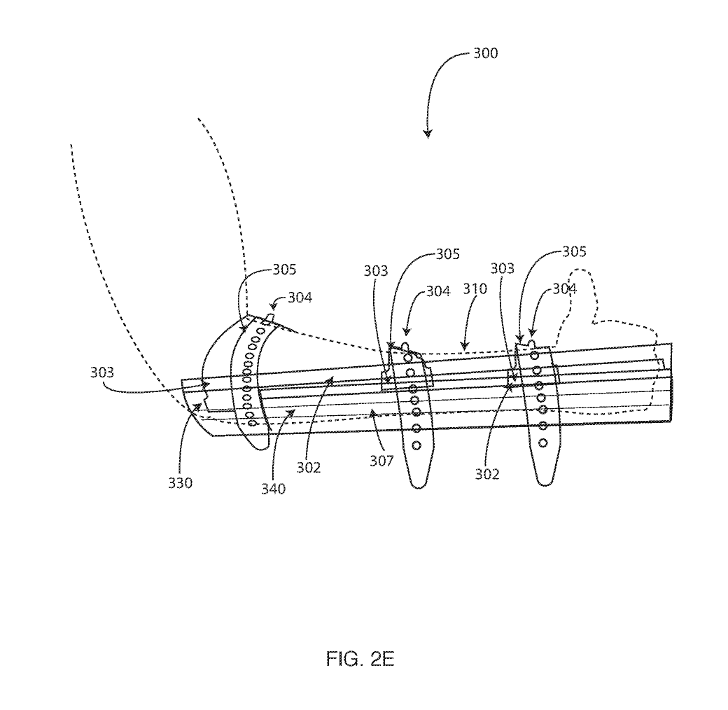

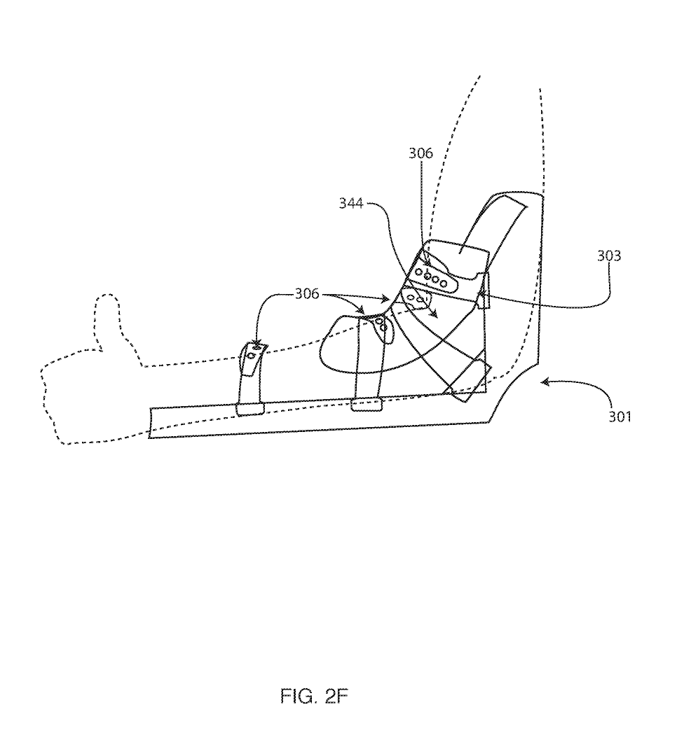

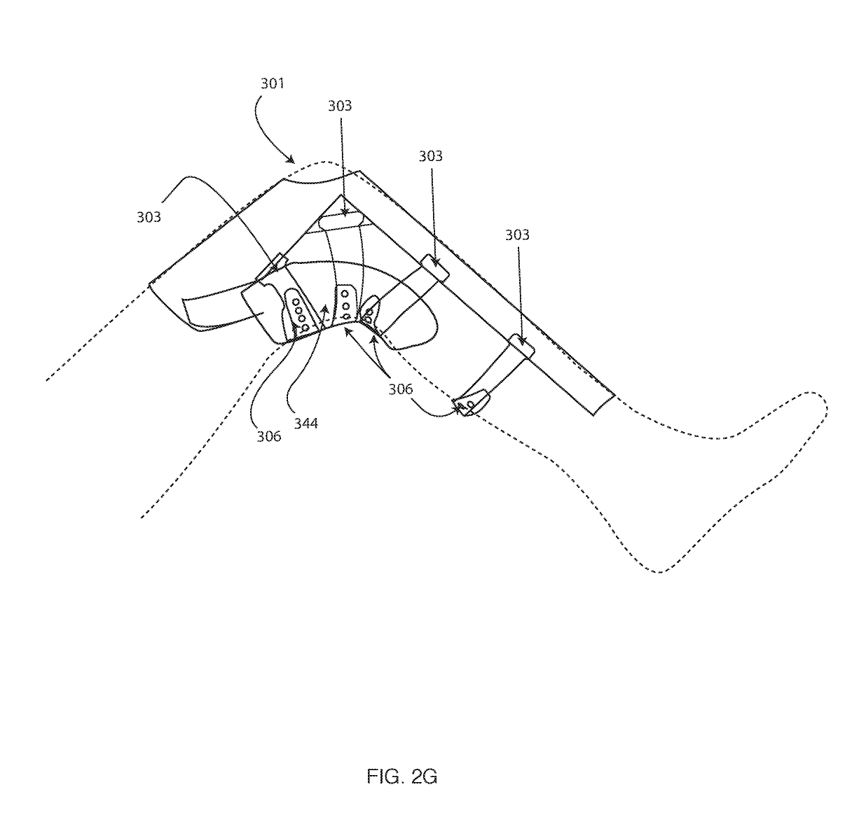

Referring to FIGS. 1A and 1B generally and collectively, the splint 300 includes an expandable support structure 310 formed by a proximal portion 330 and a distal portion 340. The distal portion 340 of the support structure 310 may be slidably coupled to the proximal portion 330 by an integrated track system 307. The track system 307 may be disposed along the lateral edges of the distal portion 340 and along the rear or back surface of the proximal portion 330. The track system 307 may include rails and/or grooves on the proximal and distal portions 330, 340. The rails may pair with the grooves such that the track system 307 secures the distal portion 340 to the proximal portion 330, while allowing the distal portion 340 to slide relative to the proximal portion 330 to transition the support structure 310 between an expanded configuration and an unexpanded configuration. The rails and/or grooves may be configured by their size, shape, length, orientation, and/or any other suitable feature, to slidably couple the distal portion 340 to the proximal portion 330. For example, the track system 307 may allow the distal portion 340 to slide in continuous motion between a fully expanded configuration (see FIGS. 1A & 1B), with the splint 300 configured with the greatest possible length, and an unexpanded configuration (see FIG. 2E), with the splint 300 configured with a majority of the distal portion 340 disposed adjacent to the proximal portion 330.

Some embodiments may allow the distal portion 340 to separate from the proximal portion 330. For example, with the splint 300 in a fully expanded configuration, the track system 307 may allow the distal portion 340 to slide away from the proximal portion 330 until the proximal and distal portions 330, 340 separate. In some circumstances, it may be advantageous to employ both the distal portion 340 and the proximal portion 330 to splint a limb (e.g., when splinting an adult limb), while in other circumstances (e.g., when splinting the limb of a child or infant) it may be advantageous to separate the distal portion 340 from the proximal portion 330 and use only the distal portion 340 or the proximal portion 330 to splint the limb. Stated differently, embodiments where the distal portion 340 and proximal portion 330 may be detached from one another may allow a responder to better adjust the length of the splint to the dimensions of the injured individual. Additionally, in some embodiments, it may be advantageous to employ only the proximal portion 330 of the splint 300.

A stop 350 located at a distal end of the proximal portion 330 may be configured to limit the distal portion 340 from sliding out of engagement with the proximal portion 330. The stop 350 may be a protrusion disposed toward a proximal end of the distal portion 340 and may be configured to engage a protrusion or other structural element at structural element disposed toward a distal end of the proximal portion 330.

In alternative embodiments, the track system 307 may be configured with a locking mechanism to substantially prevent or restrict separation of the proximal and distal portions 330, 340. For example, when the splint 300 is in a fully expanded configuration, the track system 307 may prevent movement of the distal portion 340 away from the proximal portion 330. Stated differently, the track system 307 may be configured to prevent the proximal and distal portions 330, 340 from separating.

The support structure 310 of the splint 300 may be made from any suitable material. For example, the support structure 310 may be formed from or comprise metal (e.g., aluminum), plastic, acrylic, carbon fiber, or any other suitable rigid material. In some embodiments, the slidable support structure may be radiolucent to facilitate observation of the relevant limb via x-ray. Further, the support structure 310 of the splint may be more rigid in a first direction, parallel to an axis extending from the distal portion 330 to the proximal portion 340, than a second direction transverse to the first direction. Said another way, the second direction of the support structure 310 is more flexible than the first direction to allow the support structure 310 to wrap around the limb of the patient, while maintaining a support surface for the limb. This allows the edges with the rails 302 to wrap around a limb of a patient, while maintaining support of the limb in the first direction. For example, the rails 302 on each of transverse sides of the support structure 310 are a first distance apart when the support structure is in a flat configuration and the rails 302 are a second distance apart when in the support structure 310 is in a wrapped configuration around a limb, such that the second distance is less than the first distance.

As indicated above, some splints 300 may include one or more handles 308 that are configured to facilitate lifting of splint 300 and the appendage supported by the splint 300 and/or may also facilitate configuring the splint 300 in an expanded configuration. For example, the handles 308 may allow a user to grasp each of the proximal and distal portions 330, 340 and may enable the user to pull the portions 330, 340 in opposite directions, expanding the splint. For example, each handle 308 may be disposed at or along edges of each of the proximal and distal portions 330, 340 and may include a space, an opening, an indent, and/or a groove formed in the slidable support structure 310.

The slidable support structure 310 may include a plurality of slidable securing bands/straps 305 and corresponding slidable fasteners 304 (e.g., a plurality of hook-and-loop fasteners and/or fastener elements) to adjust the configuration of the slidable support structure 310 and/or facilitate securing and/or splinting a limb with the splint 300. For example, the slidable securing straps 305 and the corresponding slidable fasteners 304 may be disposed along the lateral edges of each of the proximal and distal portions 330, 340 of the support structure 310. Further, the slidable straps 305 and corresponding slidable fasteners 304 may be slidably coupled to the proximal or distal portions 330, 340 by a slidable base 303 coupled to a rail or track 302. As shown in FIGS. 1B and 1C, the rail 302 may include a raised portion 360 of the support structure 310 configured to form an extended ridge, lip, or cavity 309. The extended ridge 309 may allow a hooked portion of each of the sliding bases 303 to couple to the rail 302 and to slide along the length of the portion 330, 340 to which each sliding base 303 is coupled. When splinting a limb (e.g., leg) it may be advantageous to wrap one or more securing straps 305 around the limb and secure the straps 305 at a length based on the girth of the limb, utilizing the slidable fasteners 304. The fasteners 304 may be buckles, hook-and-loop, or any suitable fastener to secure the strap 305 around the leg.

In some embodiments, the slidable support structure 310 may be used as a support panel of a back pack or other carrying case. The slidable support structure 310 may be removed from the back pack or other carrying case as needed. When not in use, however, the slidable support structure 310 functions as the support panel for the back pack or other carrying case for transport. That is, the slidable support structure 310 has a dual function as a splint and as a support panel of a back pack or other carrying case for ease of transport.

FIG. 2A is a side view of a leg that is supported by the splint 300 of FIG. 1A in a flat configuration and an ankle/elbow/knee device 301. FIG. 2B is a side view of a leg that is supported by the splint 300 of FIG. 1A, without the ankle/elbow/knee device 301. FIG. 2C is a side view of a leg that is supported by the ankle/elbow/knee device 301 of FIG. 1A. FIG. 2D is a side view of an arm that is supported by the splint 300 of FIG. 1A in an expanded configuration. FIG. 2E is a side view of an arm that is supported by the splint 300 of FIG. 1A in an unexpanded configuration. FIG. 2F is a side view of an arm that is supported by the ankle/elbow/knee device 301. FIG. 2G is a side view of a leg bent at the knee supported by the ankle/elbow/knee device 301.

As depicted in FIGS. 2A-2G, the splint 300 may be used to splint and/or secure the limb of an injured individual to provide support to the limb. When the limb of the injured individual has been splinted, the proximal portion 330 of the splint 300 may be disposed around an upper portion of the patient's limb, while the distal portion 340 of the splint 300 is disposed around a lower portion of the patient's limb. The splint 300 may be attached to the limb of the patient in any suitable manner (e.g., via cravats or one or more slidable securing bands 305 and corresponding fasteners 304). The slidable securing straps 305 may be of a variety of sizes (e.g., longer bands for securing the splint 300 around the upper portion of the limb and shorter bands for securing the splint around a lower portion of the limb).

The ankle/elbow/knee device 301 may be used in connection with the splint 300 to support an ankle, an elbow, or a knee of the patient. For example, the ankle/elbow/knee device 301, as shown in FIGS. 2A and 2C, may be configured (e.g., bent) in an L-shaped or substantially L-shaped configuration to cradle the ankle of the injured individual. When disposed in this manner in relation to the lower portion of the injured individual's leg, the ankle/elbow/knee 301 device may be slidably fastened to the splint 300. For example, a portion of the ankle/elbow/knee device 301 may abut the lowermost portion of the splinted leg, and may be slidably coupled to the track system 307 disposed in the distal portion 340 of the slidable support structure 310. Further, when splinting the leg of a relatively short-legged individual, the ankle/elbow/knee device 301 together with the proximal and distal portions 330, 340 of the slidable support structure 310 may adjust to decrease the overall length of the splint 300 and facilitate securing and splinting the leg. Similarly, when splinting the leg of a relatively long-legged individual, the slidable support structure 310 of the splint 300 may extend increasing the length of the splint 300 and the ankle/elbow/knee device 301 may be positioned to support the ankle region of the patient. Stated otherwise, the ankle/elbow/knee device 301 may be slidably coupled to the distal portion 340 to allow the proximal and distal portions 330, 340 and the ankle/elbow/knee device 301 to each slide along rails of the track system 307 and configure the length of the splint 300 based on the length of the limb to be secured and/or splinted. A coupling member 342 may be configured to slidably engage the track system 307 on the distal portion 340 and slidably engage a similar track system or rail on the ankle/elbow/knee device 301.

The ankle/elbow/knee device 301 may include one or more slidable securing strap 306 and corresponding slidable fasteners similar to the securing straps 305 and slidable fastener elements 304 of the splint 300. The at least one slidable securing strap 306 may include a slidable base 303 to slide along a track. The at least one slidable securing strap 306 may be configured to wrap around the patient's ankle or foot to secure the ankle/elbow/knee device 301 to the foot or leg of the patient. When disposed around the ankle and attached to the distal portion 340 of the slidable support structure 310, the ankle/elbow/knee device 301 may restrict motion of the lower portion of the injured individual's leg and/or foot. A padded angle brace 344 may be positioned at a front side of the ankle between the limb and the slidable securing strap 305. The padded angle brace 344 may enhance bracing of the ankle/elbow/knee device 301.

As can be appreciated, the same slidable configurations may be utilized to secure and/or splint an elbow in a manner substantially similar to that described above. FIG. 2F shows the ankle/elbow/knee device 301 being used to support an elbow. The elbow is supported in a bent position with the padded angle brace 344 positioned within the inner surface of the elbow. The at least one slidable securing strap 306 may be configured to wrap around the patient's elbow or arm to secure the ankle/elbow/knee device 301 to the arm of the patient. When disposed around the elbow, the ankle/elbow/knee device 301 may restrict motion of the lower portion of the injured individual's arm and the injured elbow. The padded angle brace 344 may be positioned at a front side of the elbow and may enhance bracing of the ankle/elbow/knee device 301.

FIG. 2G shows the ankle/elbow/knee device 301 being utilized to secure and/or splint a knee in a manner substantially similar to that described above. The knee is supported in a bent position with the padded angle brace 344 positioned at the posterior knee. The at least one slidable securing strap 306 may be configured to wrap around the patient's knee or leg to secure the ankle/elbow/knee device 301 to the leg of the patient at the knee. When disposed around the knee, the ankle/elbow/knee device 301 may restrict motion of the lower portion of the injured individual's leg and/or knee.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub routines or only a portion of a method described herein may be a separate method within the scope of this disclosure. Stated otherwise, some methods may include only a portion of the steps described in a more detailed method.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

The following are examples of further embodiments of the disclosed technology.

Example 1 is a splint for supporting or immobilizing a limb of a patient, the splint comprising a proximal portion formed of material that is more flexible in a first direction than a second direction transverse to the first direction, the proximal portion to bend around the limb along the first direction and to support and stabilize at least a portion of the limb along the second direction; and a distal portion formed of material that is more flexible in the first direction than the second direction, the distal portion to bend around the limb along the first direction and to support and stabilize at least another portion of the limb along the second direction. The distal portion slidably couples to the proximal portion, such that the splint can transition from an unexpanded state to an expanded state.

Example 2 is the splint of example 1, wherein the distal portion slidably couples to the proximal portion via a track system. The track system includes a first set of rails disposed on the proximal portion of the splint, and a second set of rails disposed on the distal portion of the splint to pair with the first set of rails such that the track system slidably couples the distal portion to the proximal portion.

Example 3 is the splint of examples 1 and 2, wherein the track system further includes a stop to prevent the distal portion from disengaging from the proximal portion.

Example 4 is the splint of example 3, wherein the stop includes a protrusion disposed toward a distal end of the proximal portion that engages with a protrusion disposed toward a proximal end of the distal portion to prevent the distal portion from disengaging with the proximal portion.

Example 5 is the splint of any one of examples 1-4, wherein the track system further includes a locking mechanism to prevent the distal portion from separating from the proximal portion.

Example 6 is the splint of any one of examples 1-5, wherein the proximal portion and the distal portion are detachably coupled.

Example 7 is the splint of any one of examples 1-6, further comprising a plurality of securing straps to secure the proximal portion and the distal portion to the limb of the patient.

Example 8 is a splint system including the splint of any one of examples 1-7 and an L-shaped device for immobilizing one of an ankle, an elbow, and a knee on a limb that is supported by the splint.

Example 9 is the splint system of example 8, wherein the L-shaped device is slidably couplable to the distal portion of the splint.

Example 10 is a splint for supporting or immobilizing a limb of a patient, the splint comprising a proximal portion, having a distal end, a proximal end, and two transverse sides perpendicular to the distal end and the proximal end, the proximal portion transitionable from a flat configuration to a wrapped configuration to support the limb of the patient. The flat configuration of the proximal portion includes the transverse sides spaced a first distance apart and the wrapped configuration of the proximal portion includes the transverse sides spaced a second distance apart. The second distance is less than the first distance. The splint also comprises a distal portion, having a distal end, a proximal end, and two transverse sides perpendicular to the distal end and the proximal end, the distal portion transitionable from a flat configuration to a wrapped configuration to support the limb of the patient. The flat configuration of the distal portion includes the transverse sides of the distal portion space a third distance apart and the wrapped configuration of the distal portion includes the transverse sides spaced a fourth distance apart. The third distance is less than the fourth distance. A track system is also included in the splint to slidably couple the distal portion to the proximal portion such that the splint can transition from an unexpanded state to an expanded state.

Example 11 is the splint of example 10, wherein the track system includes a first set of rails mounted to the proximal portion parallel to the transverse sides and a second set of rails mounted to the distal portion parallel to the transverse sides to pair with the first set of rails such that the track system slidably couples the distal portion to the proximal portion.

Example 12 is the splint of either example 10 or 11, wherein the track system further includes a stop to prevent the distal portion from disengaging with the proximal portion.

Example 13 is the splint of any one of examples 10-12, wherein the track system further includes a lock mechanism to prevent the distal portion from disengaging with the proximal portion.

Example 14 is the splint of any one of examples 10-13, further comprising a plurality of securing straps to secure the proximal portion and the distal portion to a limb of a patient.

Example 15 is the splint of example 14, wherein each of the plurality of securing straps is slidably coupled to one of the proximal portion and the distal portion.

Example 16 is a splint for supporting or immobilizing a limb of a patient, the splint including a proximal portion formed of a material that is bendable in a first direction to wrap the proximal portion around the limb and to configure the proximal portion to be rigid in a second direction transverse to the first direction to support and stabilize at least a portion of the limb, and a track system to slidably couple a distal portion to the proximal portion such that the splint is transitionable from an unexpanded state to an expanded state.

Example 17 is the splint of example 16, further comprising a plurality of securing straps to secure the proximal portion to the limb of the patient.

Example 18 is the splint of example 17, wherein each of the plurality of securing straps is slidably coupled to the proximal portion.

Example 19 is the splint of example 18, wherein each of the plurality of securing straps is slidably coupled about a rail on a surface opposite the track system.

Example 20 is the splint of example 19, wherein the rail includes a raised portion defining a ridge, lip, or cavity to receive each of the plurality of securing straps.

Example 21 is a splint for supporting or immobilizing a limb. The splint includes a proximal portion formed of a material that is more flexible in a first direction to wrap the proximal portion around the limb than a second direction transverse to the first direction to support and stabilize at least a portion of the limb. The splint also includes a first rail mounted on a surface of the splint and extending parallel to the second direction. The splint also includes a first strap securable to the first rail to secure the proximal portion to the limb of the patient.

Example 22 is the splint of example 21, wherein the first rail includes an extended portion to define an extended ridge to receive an end portion of the first strap.

Example 23 is the splint of example 22, wherein the end portion of the first strap includes a hooked portion to engage with the extended ridge and slide along a length of the first rail.

Example 24 is the splint of example 21, wherein the splint further includes a second rail mounted on a surface of the splint parallel to the second direction.

Example 25 is the splint of example 24, wherein the splint further includes a second strap securable to the second rail to secure the proximal portion to the limb of the patient.

Example 26 is the splint of example 25, wherein the second rail includes an extended portion to define an extended ridge to receive an end portion of the second strap.

Example 27 is the splint of example 26, wherein the end portion of the second strap includes a hooked portion to engage with the extended ridge and slide along a length of the second rail.

Example 28 is the splint of any one of examples 25-27, wherein the second strap removably couples to the first strap to secure the proximal portion to the limb of the patient.

Similarly, it should be appreciated by one of skill in the art with the benefit of this disclosure, that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

1. A splint for supporting or immobilizing a limb of a patient, the splint comprising:
a proximal portion formed of material that is more flexible in a first direction than a second direction transverse to the first direction, the proximal portion to bend around the limb along the first direction and to support and stabilize at least a portion of the limb along the second direction; and
a distal portion formed of material that is more flexible in the first direction than the second direction, the distal portion to bend around the limb along the first direction and to support and stabilize at least another portion of the limb along the second direction,
wherein the distal portion slidably couples to the proximal portion, such that the splint can transition from an unexpanded state to an expanded state.

2. The splint of claim 1, wherein the distal portion slidably couples to the proximal portion via a track system, the track system comprising:
a first set of rails disposed on the proximal portion of the splint; and
a second set of rails disposed on the distal portion of the splint to pair with the first set of rails such that the track system slidably couples the distal portion to the proximal portion.

3. The splint of claim 1, wherein the track system further includes a stop to prevent the distal portion from disengaging from the proximal portion.

4. The splint of claim 3, wherein the stop includes a protrusion disposed toward a distal end of the proximal portion that engages with a protrusion disposed toward a proximal end of the distal portion to prevent the distal portion from disengaging with the proximal portion.

5. The splint of claim 1, wherein the track system further includes a locking mechanism to prevent the distal portion from separating from the proximal portion.

6. The splint of claim 1, wherein the proximal portion and the distal portion are detachably coupled.

7. The splint of claim 1, further comprising a plurality of securing straps to secure the proximal portion and the distal portion to the limb of the patient.

8. A splint system comprising:
the splint of claim 1; and
an L-shaped device for immobilizing one of an ankle, an elbow, and a knee on a limb that is supported by the splint.

9. The splint system of claim 8, wherein the L-shaped device is slidably couplable to the distal portion of the splint.

10. A splint for supporting or immobilizing a limb of a patient, the splint comprising:
a proximal portion, having a distal end, a proximal end, and two transverse sides perpendicular to the distal end and the proximal end, the proximal portion transitionable from a flat configuration to a wrapped configuration to support the limb of the patient, wherein the flat configuration of the proximal portion includes the transverse sides spaced a first distance apart and the wrapped configuration of the proximal portion includes the transverse sides spaced a second distance apart, wherein the second distance is less than the first distance;
a distal portion, having a distal end, a proximal end, and two transverse sides perpendicular to the distal end and the proximal end, the distal portion transitionable from a flat configuration to a wrapped configuration to support the limb of the patient, wherein the flat configuration of the distal portion includes the transverse sides of the distal portion space a third distance apart and the wrapped configuration of the distal portion includes the transverse sides spaced a fourth distance apart, wherein the third distance is less than the fourth distance; and
a track system to slidably couple the distal portion to the proximal portion such that the splint can transition from an unexpanded state to an expanded state.

11. The splint of claim 10, wherein the track system includes:
a first set of rails mounted to the proximal portion parallel to the transverse sides; and
a second set of rails mounted to the distal portion parallel to the transverse sides to pair with the first set of rails such that the track system slidably couples the distal portion to the proximal portion.

12. The splint of claim 10, wherein the track system further includes a stop to prevent the distal portion from disengaging with the proximal portion.

13. The splint of claim 10, wherein the track system further includes a lock mechanism to prevent the distal portion from disengaging with the proximal portion.

14. The splint of claim 10, further comprising a plurality of securing straps to secure the proximal portion and the distal portion to a limb of a patient.

15. The splint of claim 14, wherein each of the plurality of securing straps is slidably coupled to one of the proximal portion and the distal portion.

16. A splint for supporting or immobilizing a limb of a patient, the splint including:
a proximal portion formed of a material that is bendable in a first direction to wrap the proximal portion around the limb and to configure the proximal portion to be rigid in a second direction transverse to the first direction to support and stabilize at least a portion of the limb; and
a track system to slidably couple a distal portion to the proximal portion such that the splint is transitionable from an unexpanded state to an expanded state.

17. The splint of claim 16, further comprising a plurality of securing straps to secure the proximal portion to the limb of the patient.

18. The splint of claim 17, wherein each of the plurality of securing straps is slidably coupled to the proximal portion.

19. The splint of claim 18, wherein each of the plurality of securing straps is slidably coupled about a rail on a surface opposite the track system.

20. The splint of claim 19, wherein the rail includes a raised portion defining a ridge, lip, or cavity to receive each of the plurality of securing straps.
